(19) United States
(12) Patent Application Publication
(10) Pub. No.: US 2021/0308496 A1
(43) Pub. Date: Oct. 7, 2021

(54) RESPIRATORY MASK

(71) Applicant: Francesco Paolo Monteleone, München (DE)

(72) Inventor: Francesco Paolo Monteleone, München (DE)

(21) Appl. No.: 17/223,280

(22) Filed: Apr. 6, 2021

(30) Foreign Application Priority Data
Apr. 7, 2020 (DE) 10 2020 204 512.7
Mar. 31, 2021 (DE) 10 2021 203 264.8

(51) Int. Cl. A62B 18/00; B01D 46/00; B01D 39/16; B01D 39/20; A41D 13/11; A61L 2/04; A61L 2/02; A61L 2/26; A61L 9/18; A62B 18/08; A62B 7/12; A62B 7/10; A62B 9/00; A62B 23/02; A62B 18/02; A62B 18/10; A62B 9/02

(52) U.S. Cl. CPC A62B 18/006; A61L 2209/14; B01D 46/0023; B01D 39/1623; B01D 39/2034; A41D 13/1107; A61L 2/04; A61L 2/022; A61L 2/26; A61L 9/18; A62B 18/084; A62B 7/12; A62B 7/10; A62B 9/00; A62B 23/02; A62B 18/08; A62B 18/025; A62B 18/10; A62B 9/02; B01D 2239/0208; B01D 2239/1216; B01D 2239/1241; B01D 2279/40; B01D 2267/40; A61L 2202/11; B01D 46/0028

(57) ABSTRACT

The multifunctional respiratory mask according to the invention for separating fine dust, pollen and/or viruses from breathing air has a shielding device that forms an outer and/or inner air chamber in the face region of a wearer and shields it from the environment against fine dust, pollen and/or viruses. In addition, a forehead bracket is provided having a housing arranged in the region of the forehead of the wearer and having two holding brackets, each formed laterally thereon, in the manner of eyeglass temples. A connecting line connects the housing with the air chamber closest to the face of the wearer, wherein at least one blower integrated into an air duct, a power supply, and a textile filter element are arranged in the housing. In addition, a metal plate filter element is arranged in the air duct, which forms a filter unit with the textile filter element, wherein the metal plate filter element and the textile filter element are arranged to be individually exchangeable in the housing and the power supply applies a voltage to the metal plate filter element by means of a switch or disconnects it therefrom and/or, when switched on, heats it to a temperature of at least 60° C.

### CROSS-REFERENCE TO RELATED APPLICATION

Priority is claimed to German Patent Application No. 10 2021 203 264.8, filed Mar. 31, 2021 and German Patent Application No. 10 2020 204 512.7, filed Apr. 7, 2020, the entire contents of each of which are hereby incorporated by reference herein.

### FIELD OF THE DISCLOSURE

The invention relates to a multifunctional respiratory mask for separating fine dust, pollen and/or viruses from breathing air.

### BACKGROUND

In the prior art, very many different respiratory masks for different purposes and conditions of use are known. The general purpose of respiratory masks is to separate pollutants or harmful components of the breathing air, usually to filter them off, so that the breathing air can be inhaled easily by a person wearing such a breathing mask without any damage to health. In general, a distinction is made between respiratory masks for medical purposes and for non-medical purposes. Respiratory masks with valves in order to regulate resistance, for example when inhaling, lie in a border region. Such masks are used in particular by athletes to train lung function in such a way that the lungs extract the greatest possible proportion of oxygen from the air made available. In this case, it is a training respiratory mask for developing lung function, which is used in particular in the athletic field to achieve top performance (see US 2013/0319420 A1).

DE 10 2016 013 718 A1 describes a respiratory half-mask. Respiratory half-masks are understood to mean those masks that cover the nose and mouth region of a wearer. In order to be able to use masks despite well-functioning filters, which cause not inconsiderable flow resistance, in particular when inhaling, it has been proposed to provide an inlet blower to support inhalation, which is supplied with power via batteries arranged in the mask holder. In order to keep filter elements clean, the filter means used in the respiratory half-mask are exchangeable.

In EP 0 536 546 B1, blower-assisted respiratory masks are also already described, which have particle or breathing gas filters. It is described that such masks can be designed as full masks or as half-masks. The provision, as is known in the prior art, of blower units in such respiratory masks is due to the fact that with increasing use due to increasing deposition of particles filtered out of the breathing air, the filter element clogs more and more and thus the flow resistance through this filter element increases. This also makes breathing more difficult, so a filter replacement is indicated. In order not to allow the exchange of filters to occur too frequently, the use of blower units, inter alia, is intended to reduce the additional resistance of gradually clogging filter elements.

DE 10 2018 005 650 A1 also describes a fine dust mask with an integrated ventilator, which consists of a forehead bracket, eyeglasses-like holding brackets arranged on the side and a respiratory mask to be slipped over the nose and mouth region, from which corresponding tube or hose connections to the filter elements and blowers in the forehead bracket are guided, from where the purified air can be fed back to the wearer. A power supply in the form of batteries is integrated in the forehead bracket so that the ventilator can be driven accordingly.

Furthermore, U.S. Pat. No. 5,701,892 A describes a multi-purpose respiratory mask which consists of a multilayered textile material and is stretched over the mouth region and nose region when it is attached to the ears. With regard to the filter materials, this respiratory mask is designed in such a way that dust, pollen and other common airborne particles can be filtered off. It is therefore a fine or fine dust filter. A medical application cannot be inferred from this publication.

Respiratory masks for medical purposes are usually subject to completely different conditions with regard to the particle size and thus the permeability of the filter materials used, so that a distinction is made between medical and non-medical areas even in multi-purpose respiratory masks.

### GENERAL DESCRIPTION

The object of the invention, on the other hand, is to provide a multifunctional respiratory mask which can be used for both non-medical and medical purposes or with which one can easily switch from one type of use to another type of use without complete conversion or without complete replacement of such respiratory masks.

According to the invention, the multifunctional respiratory mask is designed as a mask which can be used both for a medical or medical-like application and for a non-medical application. The multifunctional respiratory mask according to the invention is used to separate fine dust, pollen and/or viruses from the air to be inhaled.

The multifunctional respiratory mask has a shielding device that forms an air chamber in the face region of a wearer and shields the interior of the air chamber from the environment against fine dust, pollen and/or viruses. According to the invention, it is possible that only one air chamber is formed by the shielding device and that two air chambers are formed by the shielding device and are arranged one inside the other like a matryoshka. The air chambers are referred to in the following as inner and outer air chambers.

The multifunctional respiratory mask also has a forehead bracket having a housing arranged in the region of the wearer's forehead and having a bracket in the form of eyeglass temples arranged on each side of the housing, so that the forehead bracket has a holding bracket, preferably with a strap holder, and a connecting line on each side, for example a hose connection or tube connection, which connects the housing to the air chamber which is closest to the face of the wearer. In the event that only one air chamber is formed in front of the wearer's face, the connecting line leads from the housing directly into this air chamber, regardless of whether the air chamber is designed or referred to as an outer or inner air chamber. In the alternative case that the shielding device includes two air chambers lying one inside the other, that is to say both an inner and an outer air chamber, it is preferred that the connecting line is led through the outer air chamber into the interior of the inner air chamber.

The housing in the forehead bracket is now designed in such a way that at least one blower integrated in an inhalation channel, a power supply provided for this purpose, and a textile filter element are arranged therein. The inhalation channel is connected to the respiratory mask via the connecting line.

The basic structure of the multifunctional respiratory mask is therefore that the breathing air to be inhaled is fed via connecting lines, for example hose or tube connections, from the region of the forehead bracket in which the inhalation line with integrated ventilator or blower as well as the power supply and the filter elements provided for it are accommodated, into the inner or outer air chamber, depending on which air chamber is closer to the wearer's face or which air chamber is present. The filter elements realize several functions of this respiratory mask according to the invention.

According to the invention, a filter unit is now provided in the forehead bracket or in the housing of the forehead bracket, which consists of the textile filter element and a metal plate filter element. The metal plate filter element, like the textile filter element, is arranged in the inhalation channel, wherein both filter elements are arranged in the housing so that they can be replaced individually.

On the one hand they are exchangeable; on the other hand they can also be removed or reinstalled, depending on the desired function. In addition, according to the invention, a power supply for the blower and the metal plate filter element as well as a switch are arranged in the forehead bracket, wherein the power supply can be applied to or disconnected from the metal plate filter element by means of the switch and, when the power supply is switched on, this metal plate filter element is warmed to at least 60° C. or a voltage of, for example, 1V is applied to this.

The metal plate filter element is preferably provided with a disinfectant coating which has a virus-killing effect.

The shielding device of a multifunctional respiratory mask according to the invention preferably has a respiratory mask which rests on the face region of a wearer and covers it, which mask covers a nose region or a nose and mouth region of a wearer. In this case, the respiratory mask, in interaction with the wearer's face, forms an (inner) air chamber, the interior of which is shielded from fine dust, pollen and/or viruses.

In this embodiment, the multifunctional respiratory mask is designed as a nose mask or as a mouth and nose mask. In the event that the respiratory mask according to the invention is a respiratory mask formed as a nose mask that rests on the face region of a wearer and covers it, it preferably has a valve for exhalation so that exhalation takes place out of the mouth and through the valve to the outside. A silicone adapter is preferably provided as a face mask as a nose and mouth mask, or the respiratory mask is a common disposable mask used as a face mask. The respiratory mask designed as a nose and mouth mask, which covers the nose and mouth region and is designed in the shape of a dome, preferably also has an exhalation valve.

According to the invention, the shielding device preferably has a visor in order to shield the wearer's field of vision from the surroundings, regardless of whether or not an inner air chamber is provided by a respiratory mask resting on the wearer's face and covering it. This may be necessary, for example, if the wearer needs a splash guard against corrosive chemicals or works in an environment in which mechanical injury to the eyes, for example from chips thrown around, can occur.

The shielding device of the respiratory mask according to the invention preferably has sealing means which are arranged between the visor and the wearer and, together with the visor, shield an (outer) air chamber in the wearer's face region against the ingress or egress of fine dust, pollen and/or viruses.

In principle, a carrier can be shielded from fine dust, pollen and/or viruses in essentially three different ways by the shielding device according to the invention.

In a first case, a single, inner air chamber is enclosed by a shielding device designed as a dome-shaped respiratory mask that rests on the face region of the wearer and covers it, and is isolated from fine dust, pollen and/or viruses.

In the second case, no dome-shaped respiratory mask is used as a shielding device, which is why no inner air chamber is formed. Instead, the shielding device has a visor arranged in front of the wearer's field of vision, which is sealed off from the environment by sealing means in such a way that a single, outer air chamber is created that is shielded against the entry or exit of pollen, fine dust and/or viruses.

In the third case, the shielding device forms two air chambers. Thus, an outer air chamber is formed by a visor arranged in front of the face region of a wearer and sealing means arranged between the visor and the wearer. A dome-shaped respiratory mask, which rests on the face region of the wearer, is arranged in the air chamber, so that a second, inner air chamber is created in the first, outer air chamber. This can be preferred, for example, if the environment in which the wearer is staying is particularly heavily contaminated, so that a kind of “double floor,” such as that created by the two nested air chambers, is required. In this case, the connecting line preferably runs from the housing through the first outer air chamber into the inner air chamber formed by the respiratory mask, so that filtered air is always available to the wearer via the lines.

Regardless of the embodiment of the sealing means, the visor which at least partially shields the field of vision of the wearer is preferably attached to a visor holder. This can be implemented on the forehead bracket itself or as an additional device in the forehead region or in the entire head region of the wearer. A visor holder can also be provided on other additional components, such as a helmet, for example. In order to further increase the versatility of the multifunctional respiratory mask according to the invention, the visor holder can be designed in such a way that the visor can be folded up or to the side from its position protecting the wearer's face in front of the field of vision. In this way, the functionality and the scope of protection of the respiratory mask according to the invention can be adapted quickly and without major modifications to the respective place of use.

If the complete filter unit, consisting of a textile filter element and a metal plate filter element, is arranged in the forehead bracket, this multifunctional respiratory mask can be used to separate fine dust and pollen and other airborne particles, but also to separate or kill viruses from the breathing air.

For the use of respiratory masks for medical or medical-like purposes, the general teaching is that the filters used may have a pore size that prevents the respective particles to be filtered out being larger than the largest pore dimensions in the filter. It is understood that with such a filter the passage of even only one harmful virus or other particle could be fatal. The textile filter element is preferably designed such that airborne particles such as fine dust or fine dust in the range of 1 to 10 μm are reliably cleaned away. Viruses, especially coronaviruses, however, have a size of 10 to 130 nm. These viruses would be able to pass through a paper filter unhindered.

Surprisingly, it has now been shown that when using, in addition to the textile filter element, a metal plate filter element with a pore size that is definitely larger than the dimensions of viruses, for example, reliable cleaning of such viruses can still be achieved if this metal plate filter element is brought to a temperature that kills, for example, the viruses. It has now been shown that the virus can be reliably killed at temperatures of 60° C. The batteries or rechargeable batteries integrated in the forehead bracket to operate the blower can, if dimensioned accordingly, also be used to apply current to the metal plate filter element so that it is heated to a temperature of at least 60° C. Then it is possible, in addition to cleaning away fine dust or fine dust, not to clean away viruses, but to kill them so that the viruses cannot be spread with the expelled air, such that the essential point of a respiratory mask, which is, above all, to protect other people, is achieved. In addition to heating the metal plate filter element, it is also possible to apply a voltage, because even a voltage in the range from 1 volt can reliably kill these viruses. The application of a voltage to the metal plate has the further advantage that the particles in the air in the vicinity of the metal plate are statically charged and thus stick together to form larger composite particles through coagulation. Because of the increased particle diameter, the newly formed composite particles can also be easily separated from the air flow using filters with a relatively large pore diameter.

It would also be conceivable to integrate ultraviolet radiation, because these viruses can also be killed with ultraviolet radiation. Commercially available plates made of sintered stainless steel, which can be supplied in various pore size distributions, can be used as the metal plate filter element. Such sintered plates with pore sizes in the range from 1 to 15 μm, in particular 1 to 5 μm, are suitable for the multifunctional respiratory mask according to the invention. Due to a certain inherent rigidity, these plates can have a thickness of 2 to 3 mm. They can easily be inserted into a frame provided in the housing of the forehead bracket, wherein the metal plate filter element preferably is arranged in front of the textile filter element in the direction of flow of the inhaled air. However, there are no limits to the technical ability in the specific dimensioning of the dimensions and pore sizes. For example, it is also conceivable that the thickness of a filter plate is only a few tenths or hundredths of a mm.

The metal plate filter element preferably has a virus-killing coating which is applied to the metal plate filter element in addition to heating or for applying voltage, or as an alternative thereto.

The multifunctional respiratory protection mask is preferably designed to have an exhalation valve with a face mask adapter and to be without a filter element in its interior. The exhalation valve with a face mask adapter is preferably used in conjunction with a nose mask. The nose mask with exhalation valve with mouthguard adapter is used to prevent the exhaled air from being easily distributed in the ambient air; rather, it is released through the corresponding nose mask before expulsion through the exhalation valve via a normal paper filter additionally arranged in the mouthguard adapter guided so that the exhaled viruses, which may be in the exhaled air, are retained and are not released into the environment.

In one embodiment, the sealing means are arranged between the visor and the head, neck and/or torso of the wearer. Preferably, a fastening device is formed on the underside of the visor, to which a sealing means configured as a bib-shaped filter material or filter cloth can be attached. The end of the filter material facing away from the fastening device formed on the visor is fastened to the neck of the wearer in such a way that no exchange of fine dust, pollen and/or viruses between the wearer's field of vision and the environment can take place in the lower face region. A lateral seal by means of sealing bodies in the region of the ears creates an air chamber that is closed off from the environment.

The fastening device for the filter material can, for example, have one or more Velcro fasteners, adhesive connections, buttons or press studs, which can preferably be disconnected and reconnected as often as desired. The filter material can be attached to the neck of the wearer, for example by means of an elastic band or a chain.

In this case, the shape and the material of the filter material and the sealing body are preferably selected in such a way that the requirements that exist in each specific application, for example for different filtering tasks, are met. For example, the sealing body can be made of foam. Such a foam improves the wearing comfort of a respiratory mask according to the invention not only because of its comfortable surface and good skin compatibility, but also because of its noise-dampening property, which eliminates the noise burden for the wearer from air flowing through the connecting line.

According to a further embodiment, the connecting line, for example a hose connection or tube connection, which is arranged between the air chamber(s) and the forehead bracket and establishes the corresponding flow connection between these parts of the multifunctional respirator, is designed as a plug connection and is also permanently mounted. In the case of the plug connections, care must be taken to ensure that, on the one hand, they are easy to assemble and disassemble, but on the other hand, are also adequately sealed so that no unpurified breathing air can unintentionally escape into the environment or, for example, virus-contaminated inhaled air gets into the airways of a person wearing the nose mask.

A screen fabric in polyester and as a monofilament, which has a mesh size of preferably 0.5 to 150 μm, in particular 0.5 to 15 μm, is preferably used as the textile filter element. With this mesh size, the common fine dust or fine dust particles are cleaned away to a certain extent, so that in environments where such a high level of pollution could cause damage to health for people staying in such areas, it can be avoided. The metal plate filter element is also preferably designed as a sintered metal plate and has a filter fineness of in particular 1 to 15 μm, in particular 1 to 5 μm. This metal plate filter element is advantageous even when power is not applied from the power supply, even when cleaning pollen, for example, and can be removed from the housing of the respiratory mask according to the invention in the region of its forehead bracket, cleaned outside, and then reinserted and reused. In contrast, the textile filter element is disposed of after a period of use when approaching its useful life and is replaced by a new filter element in the multifunctional respiratory mask according to the invention.

The main components of the respiratory protection mask, such as a forehead bracket, a holding bracket and a respiratory mask, are preferably designed in the form of a nose mask or a nose-mouth mask with or without an exhalation valve. The dome-shaped mouthguard adapter made of plastic or silicone as well as the metal plate filter element can also be used individually if required. Simple production is possible, for example, by means of injection molding. Production by 3D printing is also possible. In the interest of good interchangeability, the textile filter element is also preferably designed with a frame which can be inserted into a frame receptacle of congruent shape in the housing or removed again for the purpose of exchanging or removing it from this frame plug connection.

In one embodiment, a shell-shaped helmet part designed as head protection is attached to the top of the forehead bracket and/or to the lateral holding brackets. This modular extension of the respiratory mask according to the invention provides the wearer with protection from falling objects, such as branches or building materials. The other functionalities of the mask, such as filtering breathing air, are not impaired.

The multifunctional respiratory mask preferably also has a tube-like portion which represents an extension of the inhalation channel present in the forehead bracket up to the nose, i.e. to the region of the upper lip below the nose. This tubular section of the inhalation channel extends from a holding bracket of the forehead bracket down into the air chamber formed in the interior of the respiratory mask, so that appropriately purified breathing air can be supplied to the nostrils for inhalation. Inhaling creates a negative pressure, which is why a good supply of purified inhalation air is guaranteed, even without the blower or ventilator switched on.

The multifunctional respiratory mask also preferably has a control device by means of which the power supply of the at least one blower can be controlled so that the blower can be adapted to the respective conditions with regard to the air flow to be inhaled or exhaled. However, the control device can also additionally or only supply the metal plate filter element with power, in order to bring it to the temperature required to kill viruses or to apply a voltage. The control device can also control a switch which ensures the power supply to the metal plate filter element for the purpose of killing it by temperature or current or switching off this metal plate filter element from the power supply.

The multifunctional respiratory mask also preferably has an additional nose bracket, which is attached to the lateral holding brackets of the forehead bracket, so that the nose bracket extends from one of the holding brackets over a region under the nose to the other of the holding brackets and thus counteracts slippage of the forehead bracket when worn. Such a nose bracket is useful, for example, when the wearer of the multifunctional respiratory mask according to the invention moves a lot, for example also during sporting activities. The holding brackets preferably have at least one fastening device for a fixing means, for example designed as a band or rubber, so that the multifunctional respiratory mask can also be attached to the wearer's head and cannot slip, so that the respirator can be kept under tension and thus always firmly on the wearer's face.

To avoid time-consuming putting on and taking off the mask, a pivoting device is preferably provided on the holding bracket or on the forehead bracket, by means of which the respiratory mask according to the invention or parts of the respiratory mask according to the invention, e.g. the visor, can be brought into a position above the wearer's head in a pivoting or circular movement from the position in front of the wearer's field of view. It is particularly preferred here if the fastening device, by means of which the sealing means are connected to the visor, is designed to be detachable. This makes it possible to remove the sealing means from the visor when the visor is to be folded up, possibly also in conjunction with other parts of the mask.

If such a pivoting device is provided on the mask according to the invention, it is further advantageous if the laterally arranged sealing bodies are designed in such a way that they have recesses and can therefore be guided past the connecting lines and the ears of the wearer during pivoting without blocking. In an alternative embodiment, the sealing bodies can be firmly connected to the stationary connecting lines, so that the sealing bodies remain in their position during a pivoting movement of the visor and a relative movement takes place between the sealing body and the visor.

In addition, the respiratory mask according to the invention preferably has a further connection for an external supply of purified air or oxygen. Especially when people stay in environments with large amounts of pollutants or harmful substances for a long time. During such a long time, the filters have already become saturated to a certain extent, so that their degree of elimination has deteriorated. It can therefore make sense to carry a reservoir for purified air or oxygen with you. For this purpose, the multifunctional respiratory mask according to the invention has a further connection to which air or oxygen, purified from the reservoir via appropriate metering and pressure-regulating devices, is supplied to the mask and thus to the wearer of the mask.

### BRIEF DESCRIPTION OF THE DRAWINGS

Further advantages, details and embodiments of the present invention will now be explained in detail with reference to the drawing. In the drawings,

FIG. 1 shows a complete multifunctional respiratory mask according to the invention worn by a wearer;

FIG. 2 shows a forehead bracket as an essential part of the multifunctional respiratory mask according to FIG. 1;

FIG. 3 shows, in a schematic representation, a plan view of the forehead bracket according to FIG. 2;

FIG. 4 is a representation of how a wearer wears the forehead bracket of the multifunctional respiratory mask;

FIG. 5 shows a further embodiment of a respiratory mask with a nose bracket for its additional attachment;

FIG. 6 shows a respiratory mask placed on a nose and mouth region in the form of a nose and mouth mask, which is attached to the forehead bracket (not shown) with an additional holding bracket;

FIG. 7 shows a tube section which is guided into the nose region of a wearer as an extension of the tube connection;

FIG. 8 shows a front view of a respiratory mask designed as a nose mask with an exhalation valve;

FIG. 9 shows a further embodiment of a nose mask with laterally arranged fastening openings; and

FIG. 10 shows another embodiment of a multifunctional respiratory mask.

### DETAILED DESCRIPTION

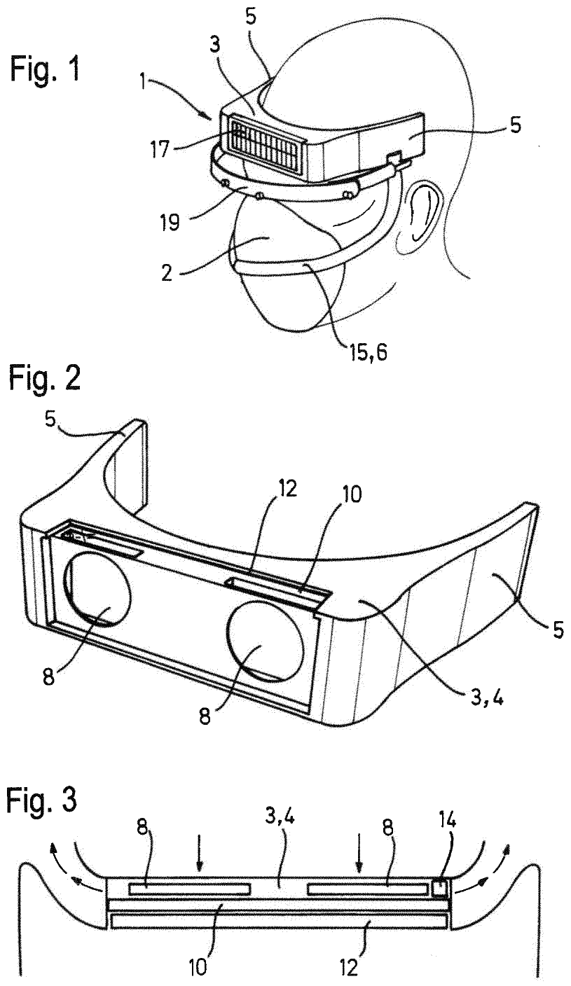

FIG. 1 shows a complete multifunctional respiratory mask 1 put on by a wearer. The main components of the multifunctional respiratory mask 1 according to the invention are a forehead bracket 3 with holding brackets 5 integrally connected to it and a housing for receiving filter elements for cleaning the breathing air and possibly other devices required for cleaning the breathing air. In the nose and mouth region, a respiratory mask 2 is put on, which is designed as a dome-shaped mouthguard adapter, possibly also in the form of a paper mask, so that its edge region rests substantially on the skin of the face or hugs it and thus forms an air chamber 22 and reliably and tightly covers the nose and mouth area to a certain extent. A nose bracket 15 is attached to the holding bracket 5, which is placed over the respiratory mask 2 and holds it so that the respiratory mask 1 consisting of forehead bracket 3, respiratory mask 2 and nose bracket 15 is reliably connected to form a unit. It is shown that the nose bracket 15 runs over the respiratory mask 2 and, so to speak, presses it firmly against the nose and mouth region of a wearer, for example by the tension from an additional fastening in the form of a band or rubber. In addition, there is a cavity in the interior of the nose bracket, so to speak as a tube connection, via which breathing air purified in the forehead bracket can be supplied to the interior of the respiratory mask for inhalation.

In addition, a removable visor holder 19 is arranged below the forehead bracket 3, spanning it in an arc. This detachable visor holder 19 makes it possible to attach a visor if necessary and to hold the attached multifunctional respiratory mask 1 in its shape, so that it does not slip and cannot easily be lost by the wearer. The holding brackets 5 are designed, based on the head size of a wearer, so that they can be pressed against the side head region from the temples to the side head region above the ears under pressing force and can also be attached to the back of the head with a band or rubber. This pressing force is dimensioned in such a way that the mask does not exert a high pressure that is uncomfortable for the wearer, but is held securely on the wearer's head to prevent slippage and loss.

An essential difference to conventional masks is that the actual respiratory mask 2, which covers the nose and mouth region, usually does not contain any filter elements, but rather, in connection with a hose connection or tube connection, feeds the interior of the respiratory mask 2, shown without exhalation valve 11, fresh, purified air for the wearer. The filter elements and other devices for optimal operation and optimal function of the multifunctional respiratory mask 1 are accommodated in the housing 4 of the forehead bracket 3. The housing 4 of the forehead bracket 3 has a grill cover 17 on its front. This grill cover 17 offers sufficient free cross-sections through which outside air is sucked in by the breathing of the wearer and is inhaled by the wearer. Two blowers or ventilators are provided behind the grill cover 17, wherein both fans 8 are provided for the inhaled air (see FIGS. 2 and 3). These fans 8 are arranged in the forehead bracket 3, expediently in at least one holding bracket 5, in which their power supply is also accommodated. Inside the housing 4 of the forehead bracket 3 and the holding bracket 5, internal channels, the inhalation channels 7 coming from the blowers 8, are provided, which lead via the corresponding tube connections or the hose connections 6 to the interior of the respiratory mask 2. Furthermore, a control device 14 is integrated into the housing 4 of the forehead bracket 3 (see FIG. 2), via which the speed and thus the volume flow of the integrated blower 8 can be regulated. A textile filter element 10 (see FIG. 3) is arranged behind the blower 8 (see FIGS. 2 and 3), which is provided essentially for filtering out fine dust, pollen, etc. In addition, in this embodiment, behind the textile filter element 10, a metal plate filter element 12 is provided in the inhalation direction, which can also be connected to the power supply consisting of batteries or accumulators. This metal plate filter element 12 can be connected to the power supply so that it is heated to a temperature in the range of 60° C. or above, so that viruses do not pass through this metal plate filter element 12 because they are killed by the high temperatures. It is equally possible to apply current to this metal plate filter element 12, which has a voltage of approximately 1 V to 1.5 V, which is sufficient that if the viruses come into contact with the metal plate filter element 12, they are killed by the current flow. An integrated switch 13 (also not shown) is also arranged in the housing 4.

It is essential in the multifunctional respiratory mask according to the invention that, depending on the intended use, either both the textile filter element 10 and the metal plate filter element 12 can be arranged in the housing 4, or it is also possible that only one of the two filter elements 10, 12 is arranged therein. If, for example, this multifunctional respiratory mask is intended mainly or exclusively for eliminating viruses, at least the metal plate filter element 12 will in any case be inserted into the corresponding frame in the housing 4. If the multifunctional respiratory mask 1 is to be used for filtering fine dust and/or pollen, then at least the textile filter element 10 will be used.

If the multifunctional respiratory mask is used to clean the breathing air of fine dust, pollen and viruses, both filter elements, i.e. the textile filter element 10 and the metal plate/filter element 12, are inserted in the respective frame within the housing 4 of the forehead bracket 3.

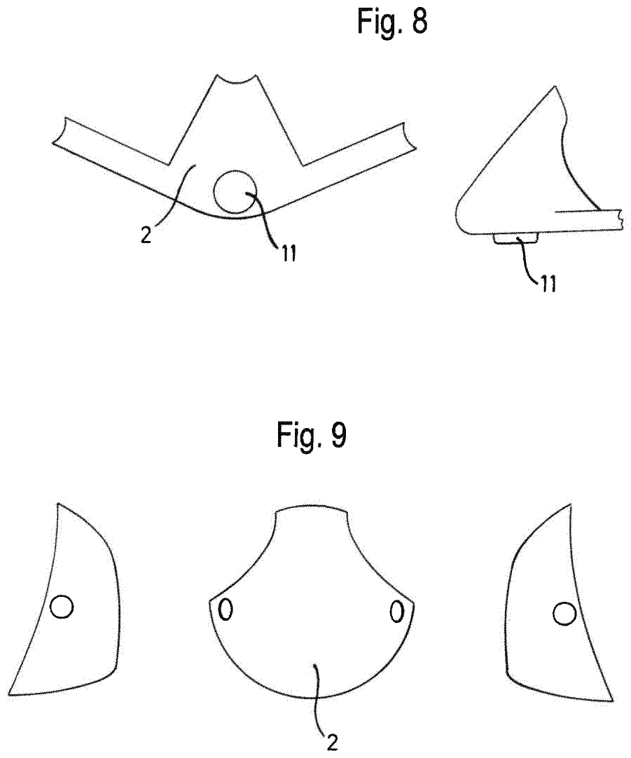

In FIG. 2, a forehead bracket 3 of the multifunctional respiratory mask 1 according to the invention is shown in a three-dimensional view. In this forehead bracket 3, the grill cover 17 (see FIG. 1) is removed. Integral with the housing 4, holding brackets 5 are provided on each side in the manner of eyeglass temples which are under mechanical tension. When the forehead bracket has been put on by a wearer, these holding brackets 5 press on the sides of the head firmly enough that the forehead bracket 3 is held, but cannot slip too easily or even get lost. Since it is the forehead bracket 3 in which the breathing air is cleaned, but not the respiratory mask 2, as is usually the case in the prior art, the forehead bracket 3 has a housing 4 for accommodating the corresponding devices. The blowers 8 built into the housing 4 are shown in principle by respective circles. The blower 8 is provided for the inhaled air, i.e. it supports the inhalation process, since the filtering of the breathable air in the filter elements 10, 12 increases its flow resistance and thus makes breathing otherwise difficult. Behind the blowers, a disk-shaped textile filter element 10, which is provided for cleaning fine dust and pollen, for example, and a metal plate filter element 12, which is arranged in the inflow direction behind the textile filter element, are conveniently inserted into the corresponding frame receptacles. By designing the filter elements as frame elements which are inserted into the frame receptacles, easy removal or easy replacement of the respective filter elements 10, 12 is possible. Preferably, a pressure sensor can also be provided which measures the pressure drop across the filter element during inhalation and, if the increase is too great, indicating that the filter is clogging, sends a corresponding signal to the control device 14, which in turn preferably gives the wearer an acoustic or optical signal which indicates that the filter element 10, 12 should be replaced or cleaned. Batteries or rechargeable batteries are housed in the interior of the holding bracket 5 and are used to supply power to the fan 8 and the metal plate filter element 12. The short inhalation channel 7 provided in the housing 4 of the forehead bracket 3 by the blower 8 and the filter elements 10 described leads via the hose connection 6 or a corresponding tube connection to the nose region of the wearer under the respiratory mask 2 (see FIG. 7).

FIG. 3 shows a basic plan view of the housing 4 of the forehead bracket 3 with the basic arrangement of the respective fan 8, behind which the textile filter element 10 and in the inflow or inhalation direction and behind it the metal plate filter element 12 are arranged. The inhaled air flows through the two filter elements 10, 12 with the support of the blower 8 for the inhaled air and from there into the tube connection 6 to the interior of the respiratory mask 2.

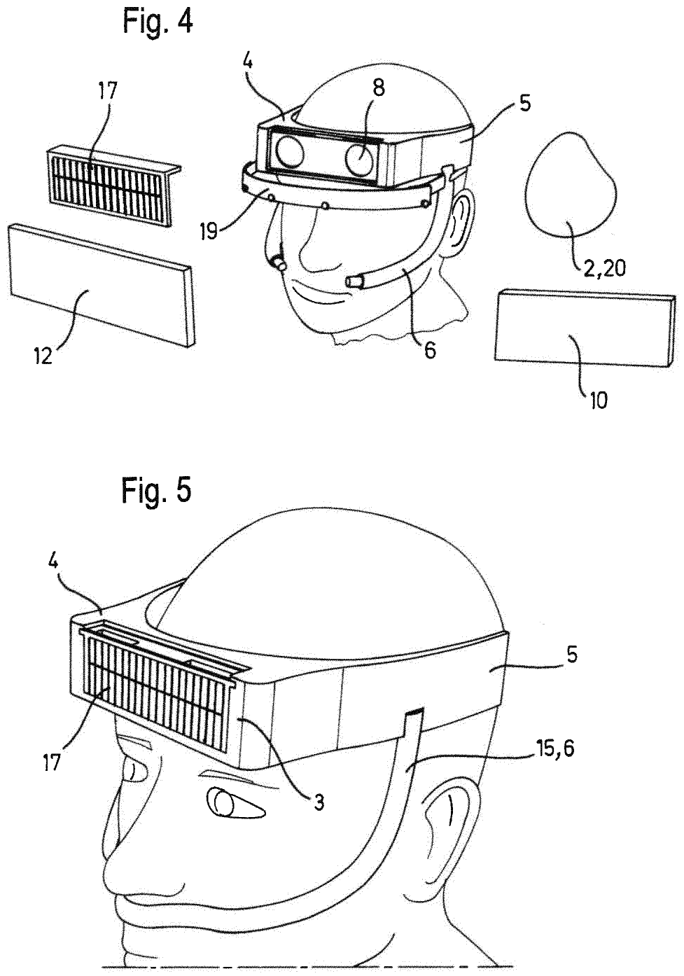

FIG. 4 again shows the forehead bracket 3 with the housing 4 and the holding bracket 5 as the main element of the multifunctional respiratory mask 1, in which the elements for cleaning away the harmful particles or viruses are arranged, from where the purified breathing air is then supplied to the interior of the respiratory mask 2 via the tube connection 6. For the sake of better illustration of the arrangement of the fan 8, the grill 17 is removed from the front bracket 3 and shown separately on the left. In the housing 4 of the forehead bracket 3, the metal plate filter element 12, which is shown on the left below the grill 17, and the textile filter element 10, which is shown schematically on the right-hand side below the respiratory mask 2, are also shown separately. On the holding brackets 5 of the forehead bracket 3, tube connections 6 are drawn, at the ends of which connecting pins are shown. Via these hollow holding brackets 5, breathing air purified in the forehead bracket 3 is supplied to the interior of the respiratory mask 2 when the connecting pins are appropriately connected to the respiratory mask 2. The respiratory mask 2 sits over the nose and mouth region of the wearer. Since it is purified inhalation air, a wearer can inhale correspondingly clean inhalation air. In this embodiment, the respiratory mask 2 is shown without an exhalation valve 11. In addition, a visor holder 19 that spans the forehead region of the wearer is attached to the respective lateral holding brackets 5 of the forehead bracket 3 and serves to additionally attach a visor in front of the face for the safety of the wearer.

Finally, FIG. 5 shows the forehead bracket 3 according to FIG. 4, but additionally with inserted grill 17 and with a nose bracket 15, which ensures an additional holder and thus securing of the forehead bracket 3 on the head of a wearer, wherein the nose bracket 15 with the respiratory mask 2 (not shown) is guided to below the nose of a wearer. Expediently, this additional nose bracket 15, in order to fulfill its holding function, is made of elastic material and is designed as a tube connection 6 so that the appropriately purified breathing air can be supplied to the interior of the respiratory mask 2 of a wearer.

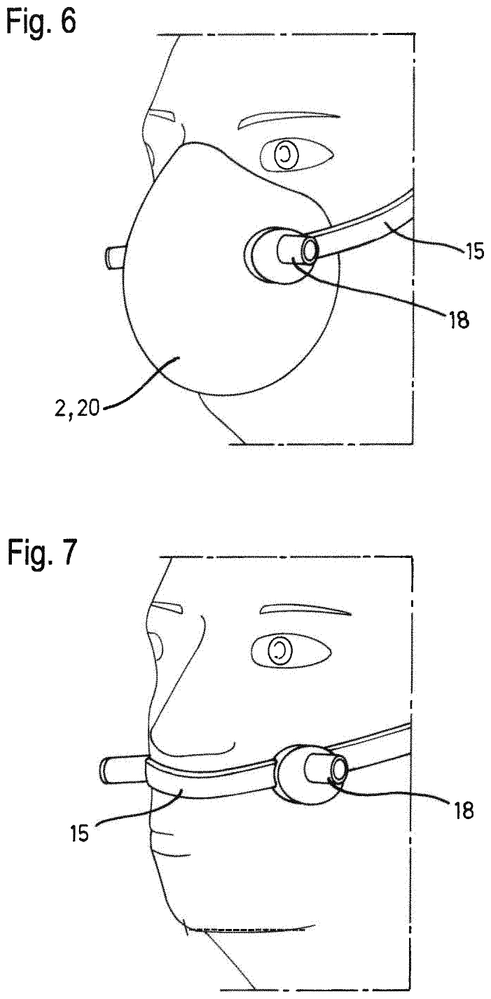

FIG. 6 shows a respiratory mask 2 applied to the nose and mouth region of a wearer. This respiratory mask 2 or this mouth protection adapter 20 is designed in the shape of a dome and with its edges substantially hugs the shape of the face of a wearer in the nose and mouth region. In the region of its attachment to the respiratory mask 2, the nose bracket 15 has a connection 18 which is connected via a plug connection to the hose connection or tube connection 6 for supplying the purified breathing air.

In FIG. 7, a hollow extension belonging below the respiratory mask 2 in its applied state to the tube connection 6 is shown, which when the respiratory mask 2 (see FIG. 6) is applied under it in the nose region of a wearer, purified air is supplied to the wearer directly into the interior of the respiratory mask therefrom. As is also shown in FIG. 6, the connection 18, at which the tube connection or the hose connection 6 is provided for supplying the inhaled air, is shown. The plug connection is designed in such a way that the hose connection or tube connection 6 is sealed after it has been plugged onto the connection 18.

The exhalation valve 11 according to FIG. 8 is designed as a flap valve, so that, when exhaling, the overpressure generated inside the respiratory mask 2 ensures that the flap of the exhalation valve 11 opens outwards so that the exhaled air reaches the outside. It is also possible that a textile filter is additionally arranged between the interior, so to speak when the respiratory mask 2 is on, and the flap opening of the exhalation valve 11, in order to hold back exhaled particles or viruses. The right of the two figures in FIG. 8 shows a side view of the nose mask.

FIG. 9 shows a nose mask with laterally attached fastening openings through which the tube connections 6 (see FIG. 4) arranged on the respective sides can be inserted and thus the nose mask is correspondingly fixed and held. The middle of the three views is a front view, whereas the two side representations each show side views.

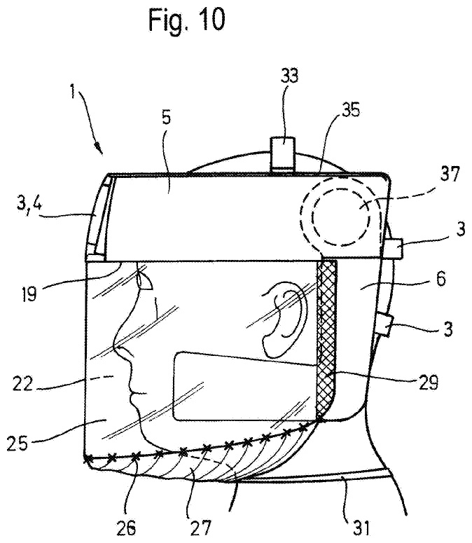

In FIG. 10, a further embodiment of a multifunctional respiratory mask 1 according to the invention is shown. The respiratory mask 1 shown has a visor 25 which is fastened to a visor holder 19 arranged on the underside of the one-piece forehead bracket 3 or holding bracket 5 and which is equipped with a fastening device 26. A filter cloth 27 is releasably fastened to the visor 25 by means of the fastening device 26. The filter cloth 27 is fixed to the neck of the wearer by an elastic band 31, for example made of a stretchable plastic. The filter cloth 27 seals the air chamber 22 enclosed by the visor 25 in front of the wearer's face in the region of the neck against fine dust, pollen and/or viruses.

In the region of the ears, the air chamber 22 is shielded from the environment by sealing bodies 29, which are arranged between the head of the wearer, the connecting lines 6 carrying filtered air, and the visor 25 so that no air exchange is possible in the region of the ears. The sealing bodies, which in this example are made of foam, additionally dampen the sound that is generated by the air flow in the connecting lines 6, so that the noise burden to which the wearer is exposed when wearing the mask is reduced.

Although the sealing bodies 29 are made airtight, the filter cloth 27 is not made completely airtight in the present example, but allows the air exhaled by the wearer to flow out, so that a slight overpressure which builds up in the air chamber 22 as the mask 1 continues to be used can be increased or decreased by adjusting the air permeability of the filter cloth 27. However, within the scope of those skilled in the art, a large number of alternative arrangements or permeabilities of the sealing elements are also conceivable, which can cause a different outflow behavior of the air from the air chamber 22.

The multifunctional respiratory mask 1 according to the invention also has a helmet receptacle 35 on the upper side of the forehead or holding bracket 3, 5, by means of which a shell-shaped helmet is attached to the bracket, so that the wearer is protected from objects falling on his head.

To avoid time-consuming putting on and taking off the mask, a swiveling device 37 is provided on the holding bracket 5 in the region of the back of the head, by means of which the visor 25 can be brought in a swiveling or circular movement from the position in front of the wearer's field of vision to a position above the wearer's head. In the present embodiment, the fastening device 26, which connects the filter cloth 27 to the visor 25, is designed to be detachable. This makes it possible to detach the filter cloth 27 from the visor 25, so that in the detached state it hangs like a bib held by the elastic band 31 in front of the wearer's chest. As a result, the visor 25 is folded upwards, wherein the laterally arranged sealing bodies 29 also advantageously are designed in such a way that they have recesses and can therefore be guided past the connecting lines 6 and the ears of the wearer during the pivoting without blocking.

Similar to a welder's mask, the embodiment of the multifunctional respiratory mask 1 shown in FIG. 10 has fixing means 33, which are preferably adjustable, by means of which the respiratory mask is reliably held on the wearer's head regardless of its pivoted position.

If the visor 25 of the multifunctional respiratory mask 1 is to be moved from the overhead position back into the position in front of the field of view and a closed air chamber 22 is formed, the visor 25 is first pivoted downwards by means of the pivoting device 37. The filter cloth 27 is then connected to the visor 25 by means of the fastening device 26, for example by means of a Velcro fastener, whereby the air chamber 22 is closed in front of the wearer's field of vision.

1. Multifunctional respiratory mask (1) for separating fine dust, pollen and/or viruses from breathing air, the multifunctional respiratory mask comprising:
a shielding device which forms an outer and/or inner air chamber (22) in the face region of a wearer and shields it from the environment against fine dust, pollen and/or viruses;
a forehead bracket (3) having a housing (4) arranged in the region of the forehead of the wearer and having two holding brackets (5), each formed laterally thereon, in the manner of eyeglass temples;
a connecting conduit (6) connecting the housing (4) to the air chamber (22) closest to the wearer's face, wherein
at least one blower (8) integrated into an air duct (7), a power supply (9) and a textile filter element (10) are arranged in the housing (4);
a metal plate filter element (12) is arranged in the air duct (7), which forms a filter unit (10, 12) with the textile filter element (10), wherein the metal plate filter element (12) and the textile filter element (10) each are individually exchangeably arranged in the housing (4); and
the power supply (9) applies a voltage to the metal plate filter element (12) by means of a switch (13) or disconnects it therefrom and/or, when switched on, heats it to a temperature of at least 60° C.

2. Multifunctional respiratory mask (1) according to claim 1, wherein the metal plate filter element (12) has a virus-killing disinfectant coating.

3. Multifunctional respiratory mask (1) according to claim 1, wherein the shielding device for forming the air chamber has a dome-shaped breathing mask (2) resting on the face region of a wearer and covering it.

4. Multifunctional respiratory mask (1) according to claim 1, wherein the shielding device has a visor (25) which shields the wearer's field of vision from the surroundings.

5. Multifunctional respiratory mask (1) according to claim 4, wherein the shielding device has sealing means for forming the air chamber, which sealing means are arranged between the visor (25) and the wearer.

6. Multifunctional respiratory mask (1) according to claim 4, wherein the visor (25) is attached to the forehead bracket (3) or to a visor holder (19).

7. Multifunctional respiratory mask (1) according to claim 4, wherein the sealing means are arranged between the visor (25) and the head, neck and/or torso of the wearer.

8. Multifunctional respiratory mask (1) according to claim 4, wherein a fastening device (26) for the sealing means designed as a bib-shaped filter cloth (27) for sealing the neck of the wearer is fastened to the underside of the visor (25) and the visor (25) is sealed in the region of the ears by means of sealing bodies (29), in particular made of foam.

9. Multifunctional respiratory mask (1) according to claim 1, wherein a shell-shaped helmet part designed as head protection can be attached to the top of the forehead bracket (3) and/or to the lateral holding brackets (5).

10. Multifunctional respiratory mask (1) according to claim 3, wherein the respiratory mask (2) is designed without filter elements.

11. Multifunctional respiratory mask (1) according to claim 1, wherein the respiratory mask (1) has a pressure-controlled exhalation valve (11) which opens when the wearer exhales.

12. Multifunctional respiratory mask (1) according to claim 1, wherein the textile filter element (10) is a mesh made of polyester as a monofilament and has a mesh size of 0.5 to 150 μm, in particular 0.5 to 15 μm.

13. Multifunctional respiratory mask (1) according to claim 1, wherein the metal plate filter element (12) is a sintered metal plate and has a filter fineness of 1 to 15 μm, in particular 1 to 5 μm.

14. Multifunctional respiratory mask (1) according to claim 1, wherein a control device (14) is arranged in the housing (4), by means of which the power supply (9) of the at least one blower (8) and/or the metal plate filter element (12) and/or its switch (13) is or are controllable.

15. Multifunctional respiratory mask (1) according to claim 1, wherein a nose bracket (15) is formed on the lateral retaining brackets (5) of the forehead bracket (3), which extends from one of the retaining brackets (5) over a region below the nose to the other of the holding brackets (5) and thus counteracts slipping of the forehead bracket (3) when worn.
